(19) United States
(12) Patent Application Publication
(10) Pub. No.: US 2008/0114391 A1
(43) Pub. Date: May 15, 2008

(54) ANEURYSM COVERING DEVICES AND DELIVERY DEVICES

(76) Inventors: Martin S. DIECK, Cupertino, CA (US); Brian B. Martin, Felton, CA (US); Maria Aboytes, Palo Altlo, CA (US)

Correspondence Address: LEVINE BAGADE HAN LLP, 2483 EAST BAYSHORE ROAD, SUITE 100, PALO ALTO, CA, 94303, US

(21) Appl. No.: 11/840,864

(22) Filed: Aug. 17, 2007

Related U.S. Application Data
(60) Provisional application No. 60/822,741, filed on Aug. 17, 2006.

(51) Int. Cl. A61M 29/00; A61F 2/06

(52) U.S. Cl. 606/200; 623/1.22

(57) ABSTRACT

Devices are provided for isolating an aneurysm from the blood vessel, particularly berry aneurysms within the cerebral vasculature. Embodiments of such devices have improved manufacturability, deliverability find isolation of the aneurysm. Delivery systems are also provided for such devices and other devices which may benefit from orientation adjustment during delivery.

### BACKGROUND OF THE INVENTION

The term aneurysm refers to any localized widening or outpouching of an artery, a vein, or the heart. All aneurysms are potentially dangerous since the wall of the dilated portion of the involved vessel can become weakened, and may possibly rupture.

A common type of aneurysm, is a brain aneurysm. Brain aneurysms are widened areas of arteries or veins within the brain itself. These may be caused by head injury, an inherited (congenital) malformation of the vessels, high blood pressure, or atherosclerosis. A common type of brain aneurysm is known as a berry aneurysm. Berry aneurysms are small, berry-shaped outpouchings of the main arteries that supply the brain and are particularly dangerous since they are susceptible to rupture, leading to often fatal bleeding within the brain. Brain aneurysms can occur at any age but are more common in adults than in children.

A variety of devices have been developed to cover such aneurysms, including stentlike devices having a one-sided covering or patch to cover the opening of the aneurysm along the blood vessel. However, such devices are often difficult to construct and deploy. In particular, these one-sided coverings need to be correctly oriented and deployed so as to cover the aneurysm opening. This is challenging in that the vascular anatomy preceding most aneurysms is very tortuous and long and therefore difficult to control and transmit torque for precise delivery. Therefore, improved devices for treatment of aneurysms are desired along with improved delivery devices and methods. At least some of these objectives will be met by the present invention.

### SUMMARY OF THE INVENTION

The description, objects and advantages of the present invention will become apparent from the detailed description to follow, together with the accompanying drawings.

Disclosed herein are vascular prosthesis for use in covering aneurysms or to provide other support within the vasculature. The disclosure also includes delivery systems for deploying such devices. In one variation the prosthesis comprise a body member having a first axis, the body member being axially extendable and compressible along the first axis, where the body member is coiled about a second axis to form a coiled prosthesis shape having a lumen extending therethrough to allow fluid flow, the coiled prosthesis shape having a plurality of adjacent helical turns separated by a gap, where the coiled prosthesis is radially adjustable relative to the second axis by adjusting the gap between the helical turns and the coiled prosthesis is also radially compliant relative to the second axis by extension and compression of the body member along the first axis.

In an additional variation, the body member of the prosthesis may comprise a super elastic material having a pattern that allows for expansion and compression along an axis of the body member. In additional variations, the body member can include a braided or woven tubular structure.

An additional variation of the prosthesis includes a plurality of wire members being wound about an axis to form a coil shape, the coil shape having a lumen extending therethrough to allow fluid flow, where the plurality of wire members include at least a first and a second wire members, the first wire member having a first end, a second end, and a mid-portion therebetween being wound about the axis, the second wire member having a first end, a second end, and a mid portion therebetween being wound about the axis, and where the first, ends of the first and second wire member are coupled together and the mid-portions of the first and second wire members are uncoupled.

### BRIEF DESCRIPTION OF THE DRAWINGS

FIGS. 1A to 3B illustrate variations of a coiled vascular prosthesis having undulations of the body member used to form the prosthesis.

FIG. 4A illustrates a stretched prosthesis that is fabricated by a heat-set flattened braided tube being wrapped in helical or coiled pattern.

FIG. 4B-4C illustrate a variation of a braided tube used to fabricate the prosthesis of FIG. 4A.

FIG. 4D shows the prosthesis of FIG. 4A when in a relaxed state and showing the overlapping of the turns of the braided tube.

FIG. 4E shows the prosthesis of FIG. 4D when the prosthesis is in a curved or bent configuration such that the overlapping of the turns decreases at the bend.

FIGS. 6A-6D illustrates another embodiment of a delivery system and covering device.

FIG. 7 illustrates the covering device of FIGS. 6A-6D positioned within a bifurcated blood vessel.

FIGS. 8A-8C illustrates a covering device comprised of a shape memory material having a coiled shape.

FIG. 9 illustrates a delivery system for used in delivering the covering device of FIGS. 8A-8C.

FIG. 10 illustrates an example of a covering device as in FIGS. 8A-8C positioned within a blood vessel so as to isolate an aneurysm.

FIGS. 11A-11B illustrates an example of a covering device as in FIGS. 8A-8C having a covering.

FIGS. 12A-12C illustrate a dual coil system.

FIGS. 12D-12E illustrates additional variations of the dual coil system comprising a plurality of coiled wires that are joined at the ends of the device but diverge towards a center of the device.

FIGS. 13A-13B, 14, 15 illustrate an embodiment of a covering device comprising a stent having a coil shape and a covering.

### DETAILED DESCRIPTION OF THE INVENTION

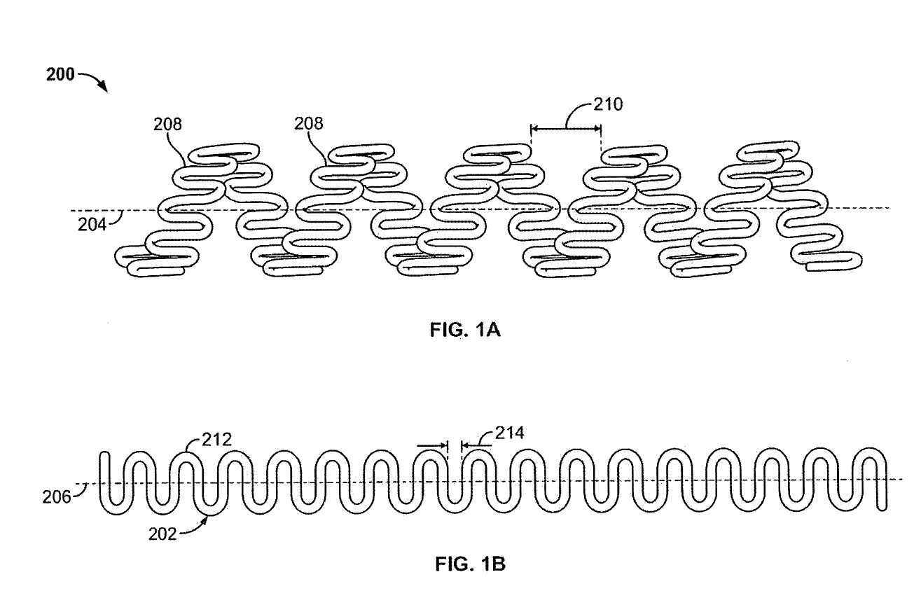

FIG. 1A illustrates a first variation of a coiled vascular prosthesis 200. As shown, the coiled prosthesis 200 comprises a body member 202 that is coiled about an axis 204. FIG. 1B shows the body member 202 prior to being coiled into a shape of a coiled prosthesis. As this variation illustrates, the body member 202 includes a series of undulations or a sinusoidal shape 212. Accordingly, the body portion is able to expand or contract along an axis of the body portion 206. Because of this feature, the coiled prosthetic shape 200 exhibits an improved compliance as compared to a simple coil when set within the vasculature.

For instance, because of its coiled arrangement, the coiled prosthetic shape 200 can increase from a small diameter configuration (e.g., by being wound tightly about a catheter, guidewire, or mandrel) for delivery to a location within the vasculature. Ultimately the coiled prosthesis 200 is deployed and assumes a larger profile. Upon deployment, the coiled prosthesis expands, or is expanded, at the target site. In such a configuration, the gap between the turns 208 increase or decrease as the coiled prosthesis 200 expands or reduces in diameter. However, because the body member 206 is able to expand or contract longitudinally relative to its axis 206, the coiled prosthesis 200 includes another degree of compliance. For example, the undulations of the body portion 202 can expand or contract, when the body portion 202 is wrapped in the coiled prosthetic shape without significantly affecting the gap between adjacent turns 208 of the prosthesis. In such a case, the spacing 214 between the undulations 212 (undulation gaps 214) increase or decrease on respective expansion or contraction longitudinally relative to its axis 206.

The prosthesis may be non-resilient, e.g., malleable, thus requiring the application of an internal force to expand it at the target site. Such an expansive force can be provided by a balloon catheter. Alternatively, the prosthesis can be self-expanding. Such self-expanding structures are provided by a temperature-sensitive superelastic material, such as Nitinol, which naturally assumes a radially expanded condition once an appropriate temperature (e.g., body temperature) has been reached. Another type of self-expanding structure uses resilient, material, such as a stainless steel, titanium, or superelastic alloy, and forming the body segment so that it possesses its desired, radially-expanded diameter when it is unconstrained, e.g., released from radially constraining forces a sheath. To remain anchored in the body lumen, the prosthesis will remain partially constrained by the lumen. The self-expanding prosthesis can be delivered in its radially constrained configuration, e.g. by placing the prosthesis within a delivery sheath or tube and retracting the sheath at the target site. Such general aspects of construction and delivery modalities are well-known in the art and do not comprise part of the present invention.

FIG. 1C shows a graft material or other covering 216 about the coiled prosthesis 200. Naturally, the covering 216 should allow for expansion and contraction of the prosthesis 200 as noted above. The covering 216 can be placed about a portion of or the entire prosthesis 200.

FIG. 1D shows another variation of the covering 216 placed about the body member 202 rather than the entire coiled shaped prosthesis 200. Again, the covering can be placed on a portion or on the entire body member 202 depending on the desired application. In addition, a single device can have a combination of the configurations shown in FIGS. 1C and 1D.

The grafts or coverings 116 for use with the present devices can be porous PTFE or ePTFE. In those cases where the graft material 116 is sealed to the body member or coiled prosthesis by a variety, the sealing may occur for example, by using an adhesive or by placing a suitable heat seal material, such as FEP (fluorinated ethylene propylene) or other thermoplastic materials, between layers of the material 116 that sandwich the body member 202 or prosthesis 200. In which case, application of heat and pressure completes the seal. In addition, a direct bond of the material to itself, via a process known as sintering, may be employed. Other methods for sealing the material could also be used. Coiled stent graft 122 includes a number of spaced apart turns 128 defining a generally helical gap 130 therebetween.

FIG. 2A illustrates another variation of coiled prosthesis 200 formed from a body member 202 as illustrated in FIG. 2B. In this variation, the body member 202 comprises undulations 212 to form a “zig-zag” pattern. As with the above variations, the body member 202 is expandable and compressible with respect to an axis of the body member 206 to provide the coiled prosthetic shape 200 with an additional degree of compliance apart from increasing or decreasing the gap 210 between turns 208 of the prosthesis 200. Clearly, the edges shown in the prosthesis of FIG. 2A can be made atraumatic by covering or coating with a graft material or polymer. Alternatively, all or some of the edges can be made rounded to reduce unwanted trauma to the vasculature.

FIG. 3A shows another variation of a coiled prosthesis 200 formed from the body member 202 illustrated in FIG. 3B and having a closed or crossed cell configuration. As shown, the body member 202 includes a series of undulations 212 that define a cell 218. As with the variations shown above, the body member 202 can expand and compress with respect to an axis of the body member 206 to provide the coiled prosthetic shape 200 with an added degree of compliance. As with other variations, the edges shown in the prosthesis of FIG. 3A can be made atraumatic by covering or coating with a graft material or polymer. Alternatively, all or some of the edges can be made rounded to reduce, unwanted trauma to the vasculature. In addition, the ends of the prosthesis 200 can have legs 240 to reduce the possibility of undesired trauma caused by the end of the prosthesis in a vessel.

The body members 202 shown above may be fabricated from a shape memory alloy (e.g., a super-elastic alloy) where the body member 202 is cut or formed to form the desired pattern either in sheet material that, is subsequently heat set into a spiral, or originally cut from tubular stock as shown in FIG. 2A. The same holds true for the implant in FIGS. 3A and 3B. The variations shown in FIG. 1 can also be so constructed. In such case, the highly rounded profile can be obtained by aggressive electro-polish (EP). Otherwise, it can be constructed using bent or heat-set wire stock.

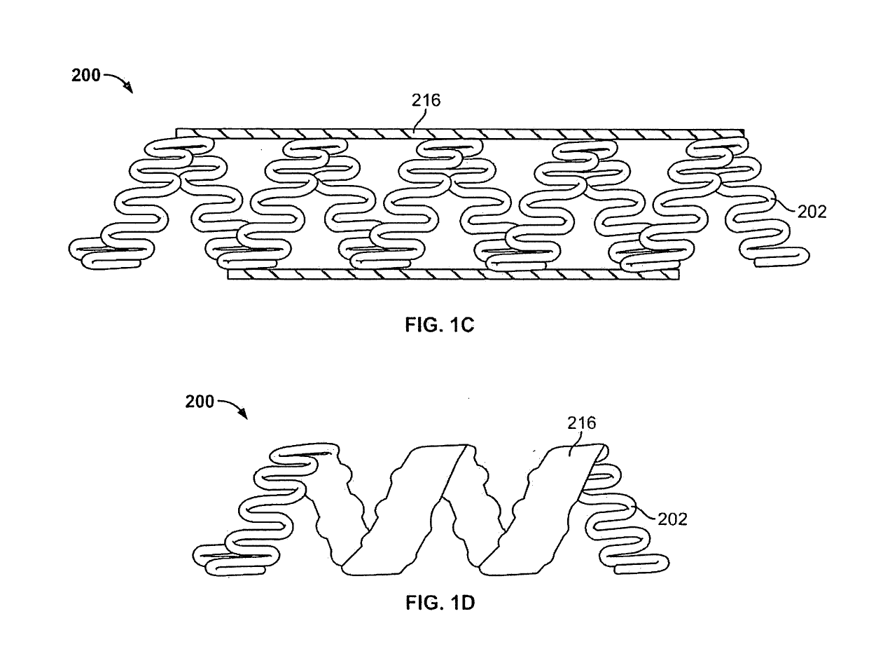

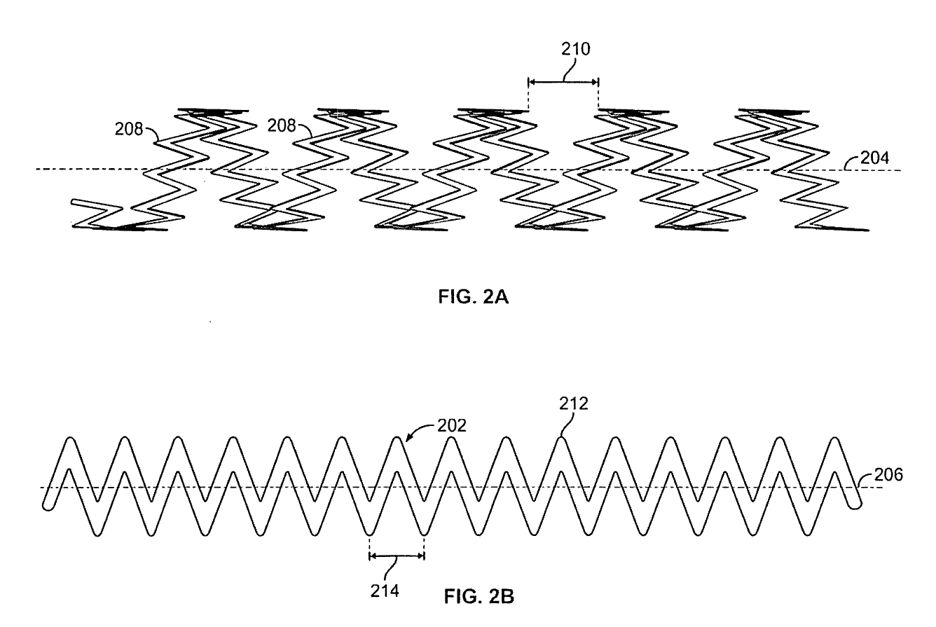

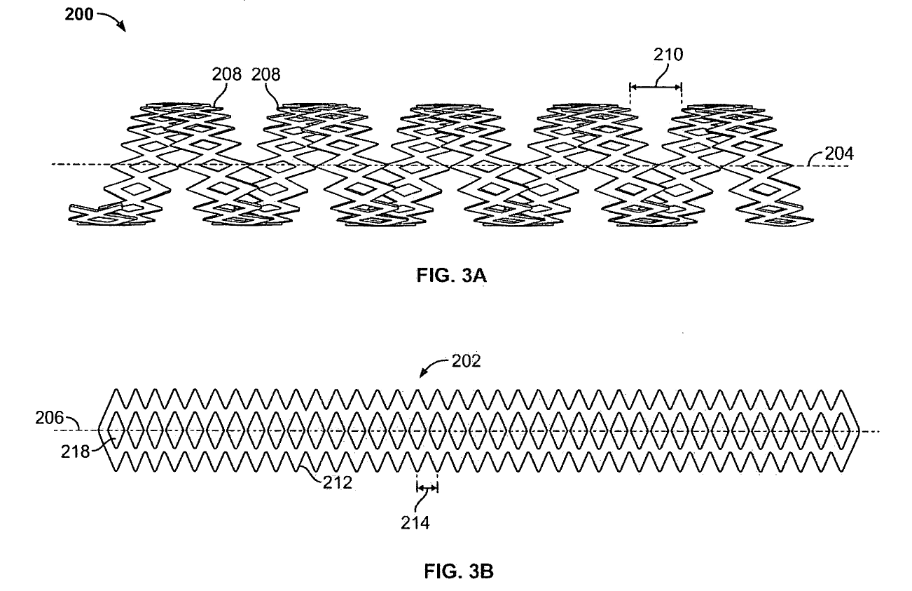

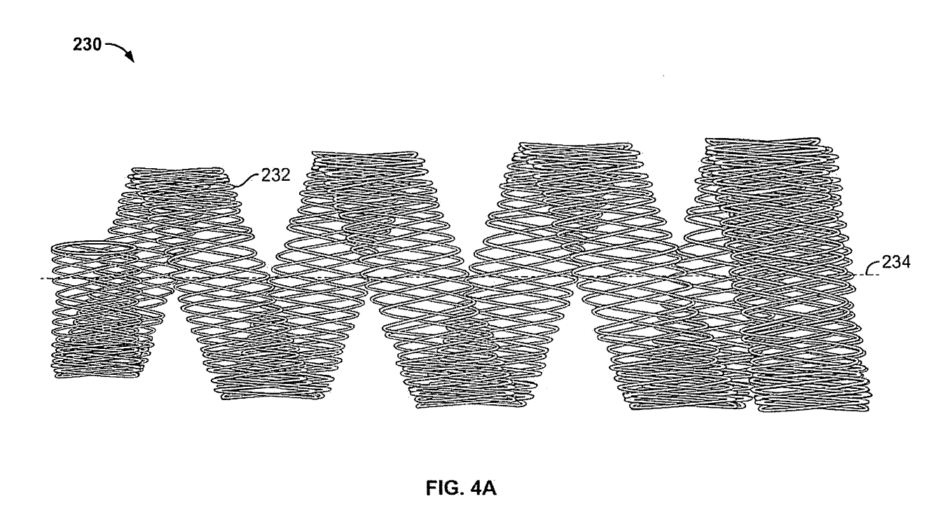

FIG. 4A illustrates another variation of a coiled vascular prosthesis 230 formed from a braided or woven tubular body member 232. FIG. 4A shows the coiled vascular prosthesis 230 as being extended for purposes of illustration. To form the prosthesis 230, a braided or woven tube 232 is helically wound about an axis 234. It may be first flattened e.g., by heat setting, or just simply be wound flat on an appropriately shaped mandrel. FIG. 4B shows a variation of a tube 232 that is ultimately forms the body member of the device shown in FIG. 4A. As shown, the tube 232 is formed about its own axis 236. FIG. 4C shows flattening of the tube of FIG. 4B. After flattening of the tube, it may then be helically configured. However, the tube 232 may be formed with an elliptical or other cross section without being fully compressed. The individual wires forming the tube 232 can be shape memory or super elastic wires. FIG. 4A shows the end of the prosthesis as being capped 242. For example, the ends of the wires can be wound by a single filament or wire.

As shown in FIG. 4D, the tube 232 of FIG. 4C may be wrapped about an axis 234 such that adjacent turns 236 of the tube 232 overlap one another at joining edges 238. One benefit of this overlapping of adjacent turns is shown in FIG. 4E. Here, when the prosthesis is bent, the turns 236 spread apart on a side of the device that is opposite to the bend. The spreading of the turns 236 decreases the overlap 238 so that the adjacent turns 236 eventually abut one another. Such a feature is useful when the prosthesis is placed within curved vasculature where the adjacent spread/flatten out rather than group together. While not shown, the prosthesis may also be configured so adjacent turns of the coil merely abut or are in close proximity to one another. Such an arrangement can offer an improved tissue interface.

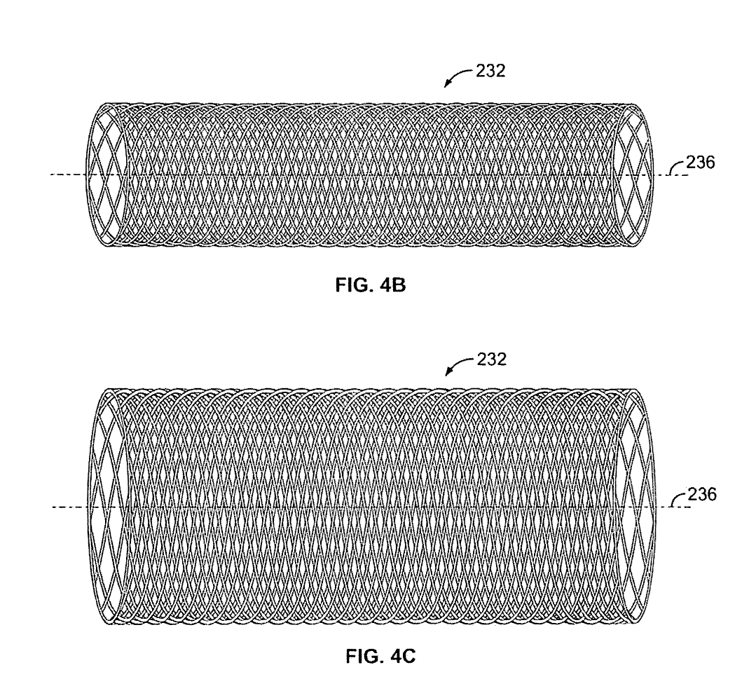

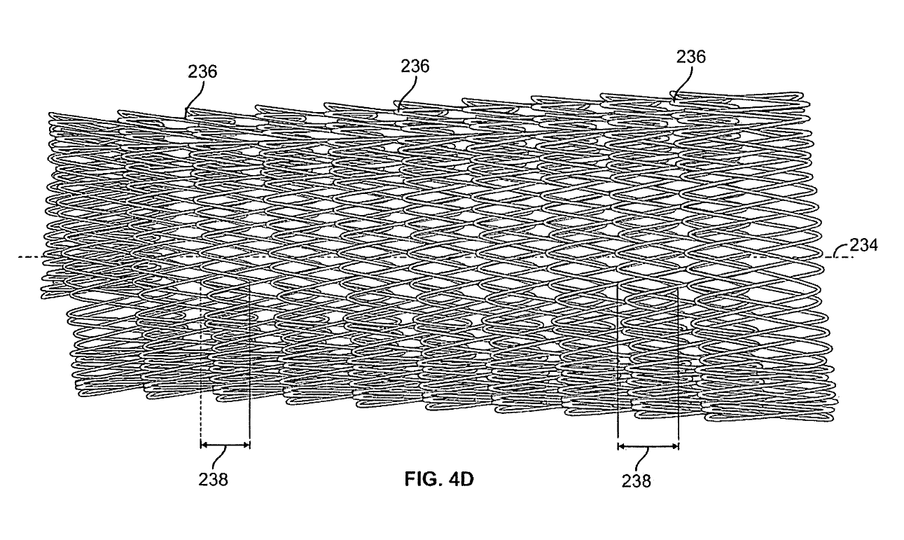

FIGS. 6A-6D illustrate an embodiment of a delivery system and covering device for use with prosthesis described herein. Referring to FIG. 6A, the delivery system 130 comprises a delivery catheter 132 and the covering device comprises a stent 136. In this illustration, the stent 136 is in a collapsed coiled shape, however any suitable shape, such as those described above or any other coiled shape, may be used. During delivery, the stent 136 is collapsed onto a distal portion of the delivery catheter 132 to allow for advancement of the catheter 132 through a blood vessel V to an aneurysm A. Referring to FIG. 6B, the stent 136 is then partially expanded and a lumen 338 is advanced from the delivery catheter 132 towards the aneurysm A. Alternatively, the lumen 138 could be in fixed position and simply utilized at the time of need. Referring to FIG. 6C, a sheet or disc is then extended from the lumen 13 8 so as to isolate the aneurysm A forming the covering 18. The covering 18 is supported by the partially expanded stent 136. The covering device 24 may be rotated by any of the mechanisms described herein to desirably position, the covering 18 over the aneurysm A. Note that the stent 136 can be partially expanded in an incremental fashion a number of times to facilitate proper placement; in this manner the stent can be expanded to bring the covering 18 close enough to the aneurysm to allow for proper evaluation of position, yet still allow rotational movement to optimize positioning. Once, desirably positioned, the stent 136 may be fully expanded within the blood vessel V which holds the covering 18 is place, as illustrated in FIG. 6D. The delivery system 130 may also be used to deliver such a stent 136 to an aneurysm A at a bifurcated blood vessel V, as illustrated in FIG. 7.

Most currently available conventional stents, whether neurological, cardiac, or peripheral in application, transform from a collapsed state for delivery to an expanded state for application. In most, or possibly all, cases, the collapsed state is a slightly diminished version of the expanded state but is not substantially different in shape or form. For example, the collapsed state is slightly smaller in outer diameter than the expanded state but perhaps not substantially different in other aspects. In addition, the collapsed state is limited in how small it can be (i.e. outer diameter cannot be smaller than a certain percentage of the expanded state outer diameter), and thus the ability to deliver stents in the collapse state to small vessels, such as neurological vessels, is limited. Therefore, the present invention provides a covering device 24 which has a different shape when in its collapsed state, allowing for a smaller cross-sectional diameter.

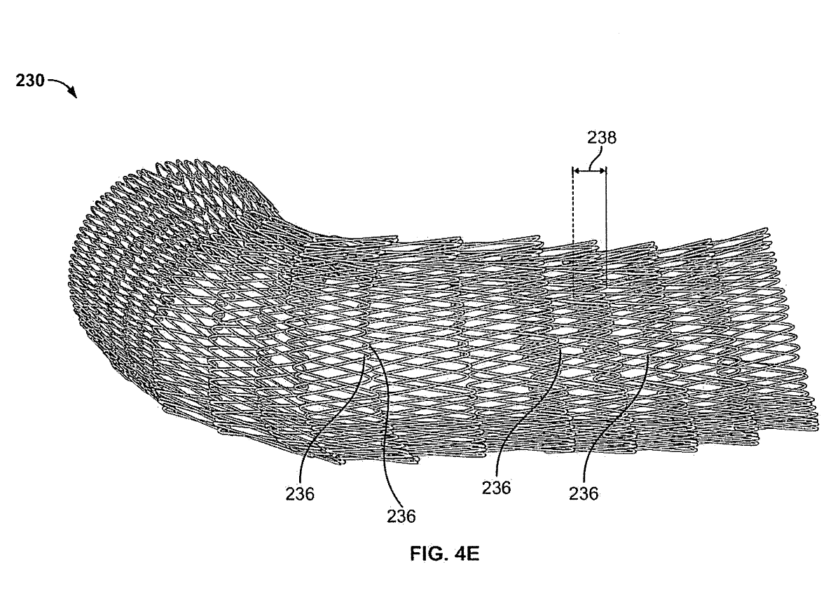

An example of such a covering device 24 is illustrated in FIGS. 8A-8C. Here, the covering device 24 comprises a shape memory material, such as nitinol, and has a coiled shape when, in an expanded state but a substantially straight or slightly curved shape when in a collapsed or unexpended state. FIG. 8A illustrates a delivery catheter 140 having an inner lumen 142 wherein the covering device 24 is loaded within the lumen 142 in a collapsed state. As shown, the covering device 24 has a substantially straight or slightly curved shape. FIG. 8B illustrates deployment of the covering device 24 into a blood vessel V. As the device 24 is released from the lumen 142, the coil expands to fill the vessel V. FIG. 8C illustrates the device 24 deployed from the catheter 140 and fully expanded. Such deployment may be achieved by “pushing” the device 24 out of the lumen 142 with the use of, for example, a push tool or mandrel, or the catheter 140 may be retracted to expose the device 24 thereby deploying the device 24.

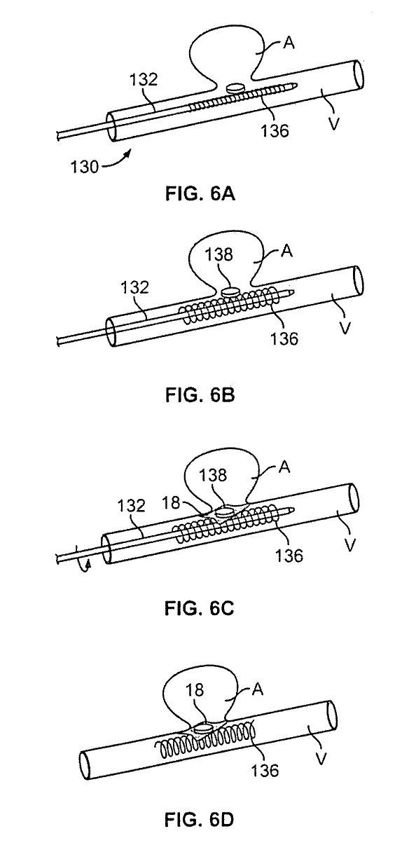

Since the embodiment of the covering device 24 illustrated in FIGS. 8A-8C has a coiled shape, the device 24 may have a tendency to rotate as it is deployed from the lumen 142. Such rotation, particularly against an inner surface of the blood vessel V, may not be desired. Therefore, in some embodiments, the delivery catheter 140 has a rotating distal section 150, as illustrated in FIG. 9. Such a rotating distal section 150 may be actively rotatable or passively rotatable. Thus, as the device 24 contacts the wall of the blood vessel V, the distal section 150 can passively rotate, allowing the device 24 to remain rotationally stable (i.e. non-rotating). Alternatively, the distal section 150 can actively rotate as the device 24 is delivered to keep the device 24 from rotating.

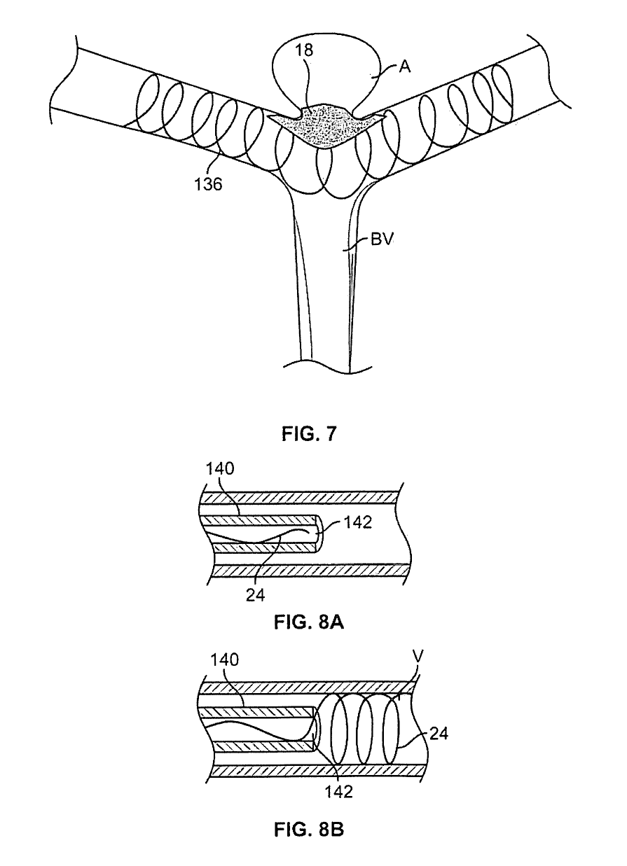

FIG. 10 illustrates an example of a covering device 24, such as illustrated in FIGS. 8A-8C positioned within a blood vessel V so as to isolate an aneurysm A. As shown, the spacing of the coils of the device 24 are adjusted manually during delivery so that the coils are denser over the aneurysm A. Such spacing may be visualized during delivery with the use of fluoroscopy. Therefore, blood flowing through the blood vessel V is isolated from, the aneurysm A by the densely spaced coils of the device 24 in the area of the aneurysm A.

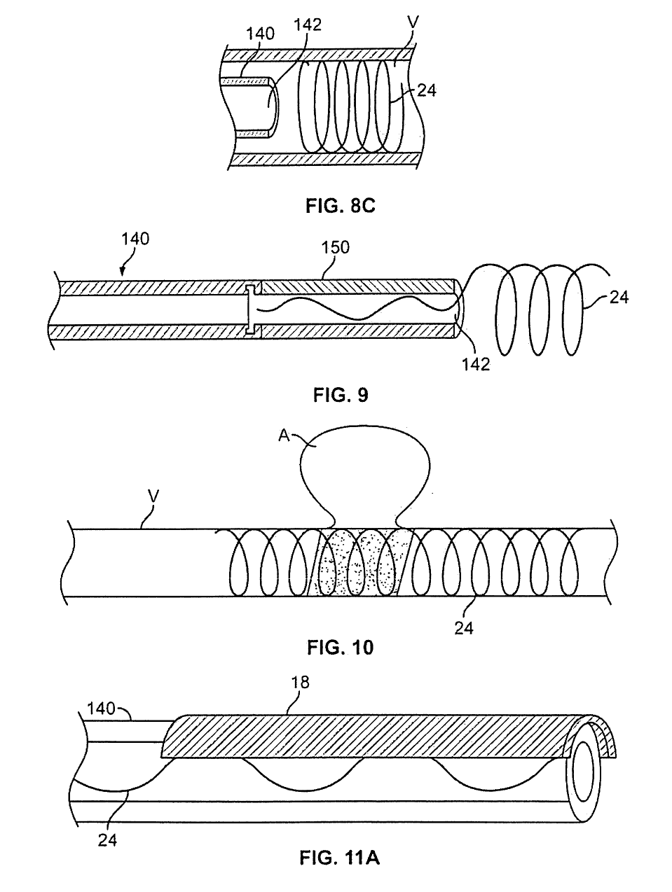

FIGS. 11A-11B illustrate an example of a covering device 24 as in FIGS. 8A-8C having a covering 18. In this embodiment, the covering 18 has the form of a strip or partially circumferential element, as shown. FIG. 11A shows the device 24 compressed within a delivery catheter 140 wherein the covering 18 is held by the delivery catheter 140. Note that covering 18 could be located on the inside or on the outside of catheter 140 prior to deployment. The covering 18 is then removed from the delivery catheter 140 by the device 24 during deployment of the device 24. The distal end of device 24 is attached to the covering 18, such that when device 24 is extended from the catheter 140 for delivery, the covering 18 is extended and deployed also. Referring to FIG. 11B, the device 24 is thus positioned within the blood vessel V so that the covering 18 isolates the aneurysm A from the blood vessel V.

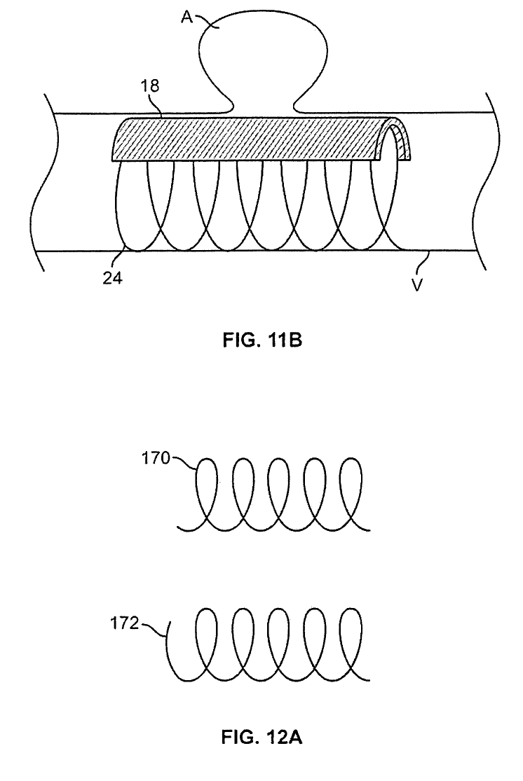

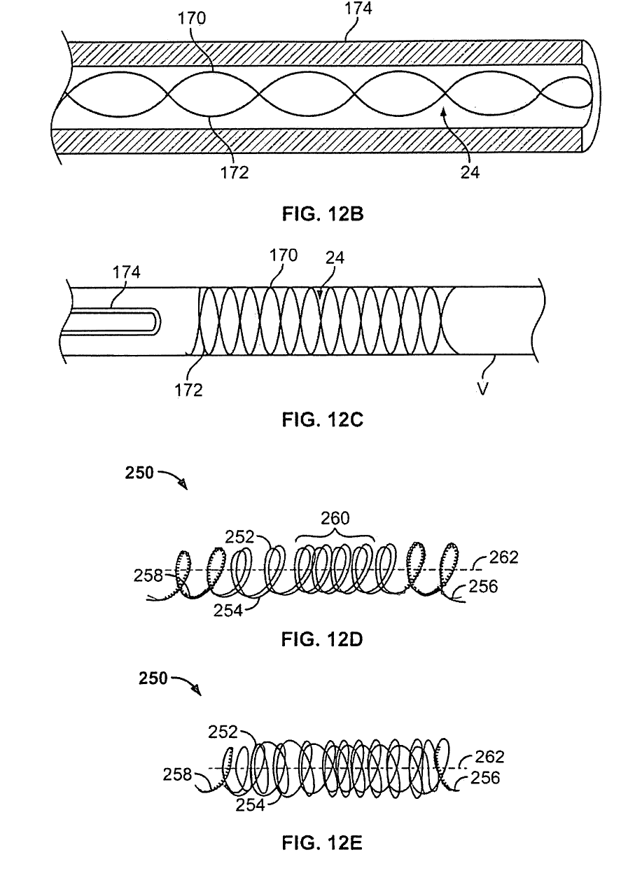

FIGS. 12A-12C illustrate another embodiment of a covering device 24 of the present invention. In this embodiment, the covering device 24 comprises a dual coil system having a first coil 170 wound in a first direction and a second coil 172 wound in a second direction which is opposite to the first, direction, as illustrated in FIG. 12A. The coils 170, 172 may he loaded into a delivery catheter 174, as illustrated in FIG. 12B. The coils 170, 172 may then be deployed into a blood vessel forming a covering device 24, as illustrated in FIG. 12C. This covering device 24 may isolate an aneurysm A in a manner such as illustrated in FIG. 33 or may utilize a covering such as in FIG. 12B. The coils 170, 172 may or may not be connected to one another at the distal end of each coil.

FIG. 12D-12E illustrate another vascular prosthesis 250 comprised of a plurality of wires 252, 254 that are wound about an axis 262 to form a coil shape. It is noted that any number of wires can be incorporated into the design but two wires are illustrated for exemplary purposes only.

As shown, the first and second wires 252,254 are coiled to form the device but one wire is helically wound about the second wire at ends 256 and 258. In alternate variations, the wires may be joined by any commonly known fastening mode. However, the wires diverge or decouple towards a center of the device 250. In some variations, a section 260 of the device 250 can be fabricated to have a high surface area or density (by high surface area or high density area it is meant that there will be smaller gaps between adjacent turns of the wire or wires). This way, the central section may serve to isolate an aneurysm, while the side sections may help anchor the implant without obstructing adjacent blood vessels.

FIG. 12D shows the plurality of coiled wires being wound in a single or similar direction such that the coils or turns are parallel. In contrast, FIG. 12E shows a plurality of wires wrapped to form a coiled prosthesis 250 but where the wires 252 and 254 are wound in opposite or different rotational directions such that the coils or turns are crossed. In this manner, better coverage maybe possible when implanting the prosthesis around a bend in the neuron-vasculature. Again, the wires 250 and 254 are joined together at the ends 256 and 258 of the device 250 and diverge or decouple towards a mid portion of the device. As with FIG. 12D, the variation of FIG. 12E can include a high density of wires or turns towards a center section of the device 250.

In one variation, the joining of the coils are sufficiently long such that the double-coil (or the portion where the coils diverge) are located only across the aneurysm neck while any perforators are better protected. The outer intertwined coil approach may also serve this purpose or merely act to efficiently connect the plurality of wires/filaments.

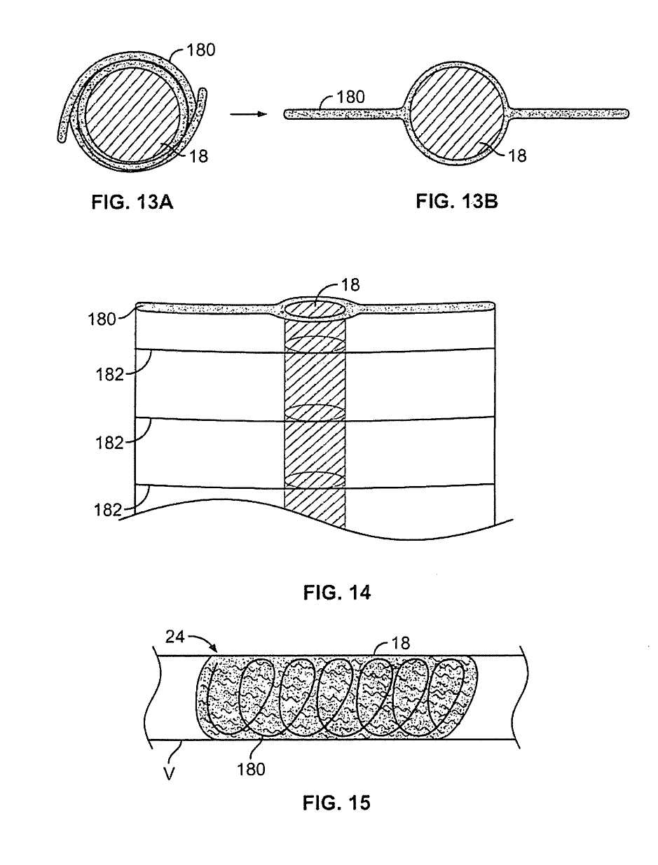

FIGS. 13A-13B, FIG. 14 and FIG. 15 illustrate another embodiment of a covering device 24 of the present invention. In this embodiment the covering device 24 comprises a stent 180, having a coil shape, and a covering 18. FIGS. 13A-13C illustrate a cross-section of one of the turns of the coil shaped stent 180. The covering 18 comprises a mesh, fabric or polymeric material that has a springiness or memory so that the covering 18 may collapse around the stent 180, as illustrated in FIG. 13A, and expand when released, as illustrated in FIG. 13B. If the covering 18 is comprised of a polymeric material, the covering 18 may be heated and shaped to impart a memory effect, such as using techniques similar to those used in forming conventional folded angioplasty balloons. Alternatively, the covering 18 may be comprised of a material that has springiness or memory provided, by one or more thin shaped memory wires 182, such as nitinol, that is set or fused with the covering 18, as illustrated in FIG. 14. FIG. 15 illustrates the covering device 24 deployed within a blood vessel V. As shown, the covering 18 extends between the turns of the coil forming a continuously covered stent 180. However, the turns of the coil may be manually spaced during delivery, similar to FIG. 10, so that the covering 18 is continuous over an aneurysm A and open near feeder vessels. Additionally, the covering 18 could be semi porous. Manually overlapping adjacent sections of covering 18 as the stent 180 is deployed could result in a final covering with different porosities, with areas of single covering being more porous and areas of overlapping covering being less porous.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended.

What is claimed is:

1. A coiled vascular prosthesis comprising:
a body member having a first axis, the body member being axially extendable and compressible along the first axis, where the body member is coiled about a second axis to form a coiled prosthesis shape having a lumen extending therethrough to allow fluid flow, the coiled prosthesis shape having a plurality of adjacent helical turns separated by a gap, where the coiled prosthesis is radially adjustable relative to the second axis by adjusting the gap between the helical turns and the coiled prosthesis is also radially compliant relative to the second axis by extension and compression of the body member along the first axis.

2. The coiled prosthesis of claim 1, where the body member comprises a sinusoidal or undulating shape.

3. The coiled prosthesis of claim 1, where the body member comprises a zig-zag shape.

4. The coiled prosthesis of claim 1, where the body member comprises a crossed-cell shape where the body member defines a plurality of closed cells.

5. The coiled prosthesis of claim 1, where at least a portion of the coiled prosthesis shape is covered by a graft material to form a tube-structure.

6. The coiled prosthesis of claim 1, where at least a portion of the body member is covered by a graft material.

7. The coiled prosthesis of claim 1, where the gap between the helical turns is consistent along a length of the coiled prosthesis shape.

8. The coiled prosthesis of claim 1, where the gap between the helical turns varies along a length of the coiled prosthesis shape.

9. The coiled prosthesis of claim 1, where the body member comprises a super-elastic material.

10. A coiled vascular prosthesis device comprising:
a tubular body member having a first end, a second end and a passage extending therethrough, where the tubular body member is set into a coil about an axis to form a coiled prosthesis shape, the coiled prosthesis shape having a lumen to allow fluid flow therethrough, the coiled prosthesis shape having a plurality of overlapping helical turns when in a straight configuration, where as the coiled prosthesis assumes a curved profile, the overlap of the helical turns located on a side of the coiled prosthesis decreases.

11. The coiled vascular prosthesis of claim 10, where tire tubular member comprises a braided tube.

12. The coiled vascular prosthesis of claim 10, where the tubular member comprises a flattened tube having an elliptical cross section.

13. The coiled vascular prosthesis of claim 10, where the tubular member comprises a super-elastic tubular braid.

14. A vascular prosthesis comprising:
a plurality of wire members being wound about an axis to form a coil shape, the coil shape having a lumen extending therethrough to allow fluid flow;
where the plurality of wire members include at least a first and a second wire members;
the first wire member having a first end, a second end, and a mid-portion therebetween being wound about the axis;
the second wire member having a first end, a second end, and a mid portion therebetween being wound about the axis;
where the first ends of the first and second wire member are coupled together and the mid-portions of the first and second wire members are uncoupled.

15. The vascular prosthesis of claim 14, where the first wire and second wire are wound in a first rotational direction.

16. The vascular prosthesis of claim 14, where the first wire is wound in a first rotational direction, and where the second wire is wound in a second rotational direction.

17. The vascular prosthesis of claim 14, where the first and second wire member comprise super-elastic alloys.

18. The vascular prosthesis of claim 14, where the coil shape comprises an increased surface area in a mid portion where the increased surface area comprises the plurality of wires.
